(19) United States
(12) Patent Application Publication
(10) Pub. No.: US 2001/0053387 A1
(43) Pub. Date: Dec. 20, 2001

(54) Benzimidazole pharmaceutical composition and process of prepatation

(76) Inventors: Yusuf Khwaja Hamied, Mumbai India (IN); Vinay G. Nayak, Mumbai (IN); Geena Malhotra, Mumbai (IN)

Correspondence Address: STEVENS, DAVIS, MILLER & MOSHER, L.L.P., Suite 850, 1615 LStreet, N.W., Washington, DC, 20036, US

(21) Appl. No.: 09/900,200

(22) Filed: Jul. 9, 2001

Related U.S. Application Data
(63) Continuation of application No. 09/424,024, filed on Feb. 25, 2000.
Application No. 09/424,024 is a 371 of international application No. PCT/GB98/01465, filed on May 21, 1998.

(30) Foreign Application Priority Data
May 23, 1997 (GB) 9710800.5

(51) Int. Cl. A61K 9/48; A61K 9/28

(52) U.S. Cl. 424/474; 514/394; 514/388

(57) ABSTRACT

A pharmaceutical composition which is a solid pellet comprising an inert core, a benzimidazole in or on the core, a moisture resistant coating around the core, the moisture resistant coating comprising at least one hydrophobic material, and an enteric coating around the moisture resistant coating.

The present invention relates to a pharmaceutical composition and to a process of preparation thereof, and more particularly to a pharmaceutical composition containing a benzimidazole.

Benzimidazole derivatives, such as omeprazole, lansoprazole and timoprazole, etc., are known potent proton pump inhibitors with powerful inhibitory action against the secretion of gastric acid (Lancet, Nov. 27, 1982, pages 1223-1224). They are used in the treatment of Zollinger-Ellison syndrome and stress related oesophagitis ulceration. The derivatives are well known and are described, for example, in EP-A-0005129.

It has been found that these benzimidazole derivatives, and in particular omeprazole, are susceptible to degradation in acid and neutral media and it is known to protect oral dosage forms by provision of an enteric coating. In this way, the active material is protected from acidic gastric juices until it reaches the site of desired release, e.g. the small intestine. Because certain enteric coatings can themselves be, or contain, acidic materials it is also often required to protect the benzimidazole from the acidity of the enteric coatings. For example, it is known to formulate the benzimidazole with an alkaline material before applying the enteric coating; it is also known to provide an intermediate coating between the benzimidazole and the enteric coating, generally the intermediate coating is selected so as to be substantially water soluble.

EP-A-0247983 describes an oral pharmaceutical preparation of omeprazole which is composed of a core material in the form of small beads or tablets containing omeprazole together with an alkaline reacting compound, the core material having one or more inert reacting subcoating layers thereon. The alkaline reacting compounds can be chosen among but are not restricted to substances such as the sodium, potassium, calcium, magnesium and aluminium salts of phosphoric acid, carbonic acid, citric acid or other suitable weak inorganic or organic acids; substances normally used in antacid preparations such as aluminium, calcium and magnesium hydroxides; magnesium oxide or composite substances, such as Al₂O₃.6MgO.CO₂. 12H₂O, (Mg₆Al₂(OH)₁₆CO₃.4H₂O), MgO.Al₂O₃.2SiO₂.nH₂O or similar compounds; organic pH-buffering substances such as trihydroxymethylaminomethane or other similar, pharmaceutically acceptable pH-buffering substances. The specification further states that if an alkaline reacting salt of omeprazole such as the sodium, potassium, magnesium, calcium or ammonium salt of omeprazole (which are described in EP-A-0124495) is used, this can be in place of or in addition to the alkaline reacting compound.

TW289733 describes a process of preparing pellets containing omeprazole. The process comprises spraying a solution comprising omeprazole, excipient, ethanol, water, ammonia and binder on an inert core; granulating; spraying an intermediate coating solution comprising excipient, ethanol, water, ammonia and binder on the granules; and providing a final outer coating.

U.S. Pat. No. 4,786,505 describes pharmaceutical preparations containing omeprazole in an alkali environment as the core material, one or more inert subcoating layers which are water soluble and an outer enteric coating.

There is however provided by the present invention pharmaceutical compositions, and processes of preparing the same, wherein an active ingredient comprising a benzimidazole can be protected from surrounding acidic media, and the compositions offer further advantages over the prior art by exhibiting extended shelf life.

According to the present invention, there is provided a pharmaceutical composition which is a solid pellet comprising an inert core, a benzimidazole in or on the core, a moisture resistant coating around the core, the moisture resistant coating comprising at least one hydrophobic material, and an enteric coating around the moisture resistant coating.

There is further provided by the present invention a process of preparing a composition substantially as described above, which process comprises providing an inert core having a benzimidazole therein or thereon, applying a moisture resistant coating around the core, the moisture resistant coating comprising at least one hydrophobic material, and applying an enteric coating around the moisture resistant coating.

Preferably, the benzimidazole in or on the core is present in an alkaline environment, and more particularly the benzimidazole is present as an intimate mixture with at least one alkali. Suitably, the alkali is ammonia or a solution of ammonia or ammonium carbonate. A process according to the present invention therefore preferably further comprises formulating the benzimidazole in an alkaline environment substantially as herein described.

It is generally preferred that an alkali employed in the present invention comprises an aqueous solution of ammonia (i.e. ammonium hydroxide) or ammonium carbonate, although it is of course also possible to use liquid ammonia or ammonia gas. In such cases, the benzimidazole may be formulated under an ammonia atmosphere, for example, and the gas may be absorbed, such as by dissolution in any aqueous material present.

In accordance with the invention, the pH of the benzimidazole-containing part of any formulation is preferably from above 7 to 10, and is more preferably in the range 8 to 9. Advantageously, an ammonia solution is used, the solution preferably containing from 20 to 40 wt % ammonia, more preferably about 30 wt % ammonia.

The invention is applicable to pharmaceutically active benzimidazole derivatives. Particularly useful such derivatives are the alkali metal salts of the benzimidazole. Examples of specific useful derivatives are omeprazole, lansoprazole, timoprazole, pariprazole and pantoprazole, in particular omeprazole.

The purpose of the moisture resistant coating employed in the present invention is to resist moisture absorption by the pharmaceutical composition, thereby extending shelf life. Suitably, the hydrophobic material of the coating is present in sufficient amount to ensure that the coating substantially repels water therefrom. The hydrophobic material can be solid or liquid, and is desirably selected from the group consisting of a polyalkylsiloxane, castor oil, mineral oil, isopropyl myristate, stearic acid, cetyl alcohol or the like. Preferably, the hydrophobic material comprises a polyalkylsiloxane, and it is particularly preferred that the hydrophobic material comprises polydimethylsiloxane.

The moisture resistant coating may further comprise at least one binding agent. The binding agent may be hydrophobic or hydrophilic. In the latter case, the binding agent is incorporated in the moisture resistant coating in an amount which ensures that the water repellent properties of the latter (as provided by the hydrophobic material substantially as described above), are substantially unaffected by the hydrophilic nature of the binding agent. The binding agent is preferably selected from the group consisting of a sugar, polyvinyl-pyrrolidone, shellac and gums, such as xanthan gum, or the like. It is especially preferred that the binding agent comprises a sugar, such as sucrose or the like.

Advantageously, a binding agent is employed in the moisture resistant coating when the hydrophobic material is a liquid. A preferred moisture resistant coating comprises an emulsion of a polyalkylsiloxane (especially polydimethylsiloxane) or an admixture thereof, with the binding agent.

The moisture resistant coating may contain one or more other conventional additives that typically aid in the process of adhesion onto the inert core.

The moisture resistant coating may also be useful in benzimidazole pharmaceutical compositions that do not have an alkaline binder. The moisture resistant coating can be typically applied by spraying, using conventional equipment. In addition to the moisture resistant coating applied directly to the inert core substantially as hereinbefore described, a further moisture resistant coating can be provided over the enteric coating.

A wide variety of conventional enteric coatings may be employed in the present invention, including for example: cellulose acetate phthalate; hydroxypropyl methyl cellulose phthalate (HPMCP); hydroxypropyl cellulose acetyl succinate; polyvinyl acetate phthalate; copolymerised methacrylic acid/methacrylic acid methyl esters, such as Eudragit L 12-5, Eudragit L 100 55 or Eudragit S 100; and mixtures thereof. The enteric coating may contain conventional plasticisers, pigments and/or dispersants, including for example polyethylene glycols, triacetin, triethyl citrate, and Citroflex, dibutyl sebacate.

The enteric coating can be applied in any suitable manner, for example in the form of an aqueous dispersion in water, or other dispersing medium, or in the form of a solution. It is preferred that a dispersion or solution of the enteric coating is treated with an alkali in order to neutralise at least part of any free acid content. The alkali may be, for example, a carbonate or hydroxide of sodium, potassium, magnesium or calcium.

According to a first embodiment of the present invention, the benzimidazole is present in the inert core. Suitably, the inert core of the pharmaceutical composition comprises a plurality of compressed granules of the benzimidazole. This embodiment is particularly useful when it is desired to provide the pharmaceutical composition in tablet form, and there is further provided by the present invention a tablet which comprises a pharmaceutical composition substantially as herein described, wherein the inert core is formed from a plurality of granules comprising the benzimidazole, which granules are compressed together to form the core.

According to the first embodiment of the present invention, the moisture resistant coating is applied around the inert core, then the enteric coating is suitably provided around the moisture resistant coating on the inert core.

According to a second embodiment of the present invention, the benzimidazole is present on the inert core. The second embodiment of the present invention is particularly applicable for the inclusion of a plurality of pellets substantially as herein before described in a capsule. The inert cores of the pellets may typically be non-pareils, and suitably provided in the form of sugar beads or sugar/starch beads. According to the second embodiment of the present invention there is therefore provided a capsule which comprises a capsule shell containing a plurality of pellets substantially as herein before described.

According to the second embodiment of the present invention, the moisture resistant coating is applied around the inert core of each of the pellets to be provided in a capsule, and the enteric coating is suitably provided around the moisture resistant coating on each of the inert cores.

Pharmaceutical compositions according to the present invention may comprise one or more additives. Examples of particularly useful additives include a solubiliser to aid solubilisation of the pharmaceutically active ingredient, and a lubricant to aid flow of the active ingredient during manufacture. The solubiliser may be, for example, a sugar, which is preferably in pulverised form. An example of a suitable sugar is sucrose. The lubricant may be, for example, starch and/or talcum. It will be appreciated that the pharmaceutical compositions of the invention may contain any one or more other additives conventionally used in the formulation of pharmaceutical compositions.

The pharmaceutical compositions of the invention may be used to treat conditions in the same manner as the prior known benzimidazole compositions. The pharmaceutical compositions may be formulated for oral, topical, parenteral or rectal administration. Oral administration is preferred.

The pharmaceutical compositions may take the form of, for example, a tablet or peltab (e.g. comprising a plurality of granules comprising a benzimidazole, together with conventional excipients, such as disintegrants and binders, compressed into a tablet) or a capsule (e.g. containing a plurality of individual pellets comprising a benzimidazole disposed within the capsule shell). Furthermore, the pharmaceutical composition may include conventional excipients.

Tablets to be employed in compositions of the invention can be made, for example, by using equipment known as a marumerizer (which is also called a spheronizer). In such cases, core ingredients, including the benzimidazole, can be extruded into the marumerizer, and converted into substantially spherical granules by a high speed rotating disk. Subsequently, the granules may be compressed by conventional means in order to form a solid core, and subsequently coated with a moisture resistant coating and an enteric coating as herein before described.

When, alternatively, the pellets comprise benzimidazole loaded onto a plurality of inert cores suitable for inclusion in a capsule, the benzimidazole can be supplied as a spray, for example. The benzimidazole may be mixed with one or more additives before being loaded on the inert cores. As described above, the additives may include, for example, a solubiliser and/or a lubricant. The inert cores can be loaded with the benzimidazole (together with any additives), and sprayed with a binder, in a centrifugal coating apparatus.

The following Intermediate Examples and Examples illustrate the invention. In each case, the active drug was omeprazole unless indicated otherwise. Whilst sucrose (sugar) is the illustrated binding agent, other binding agents such as polyvinylpyrrolidone, shellac or xanthan gum, may be used instead.

### INTERMEDIATE EXAMPLE 1

A plurality of particles containing the active drug were prepared from the following materials:

| | |
|---|---|
| Non-pareil seeds | 95.00 mg |
| Active drug | 20.00 mg |
| Sucrose | 32.00 mg |
| Corn starch | 32.00 mg |
| Talcum | 10.00 mg |
| HPMC | 1.00 mg |
| | 90.00 mg |

Water: as required.

Particles were also made of the above materials with the addition of 30% by weight ammonia solution in an amount to provide a pH of 8.0-9.0.

Initially, the active drug, the sucrose, the corn starch and the talcum were blended thoroughly to yield a dusting powder. The non-pareil seeds were loaded into a centrigual coater and then coated with the dusting powder while spraying the HPMC (hydroxypropyl methyl cellulose) solution, with the ammonia solution when used. This resulted in the production of a plurality of discrete particles containing the active ingredient. The particles so obtained were dried using conventional tray dryers/fluid bed dryers to an outlet temp. of 45° C.

### INTERMEDIATE EXAMPLE 2

A plurality of particles containing the active drug were prepared as follows:

| | |
|---|---|
| Non-pareil seeds | 95.00 mg |
| Active drug | 20.00 mg |
| Sucrose | 32.00 mg |
| Corn starch | 32.00 mg |
| Talcum | 10.00 mg |
| HPMC | 1.00 mg |
| | 190.00 mg |

Water: as required.

Particles were also made of the above materials with the addition of 3.00 mg ammonium carbonate.

Initially, the active drug, the sucrose, the corn starch, the ammonium carbonate (when present) and the talcum were blended thoroughly to yield a dusting powder. The non-pareil seeds were loaded into the centrifugal coater and then coated with the dusting powder while spraying the HPMC (hydroxypropyl methyl cellulose) solution. This resulted in the production of a plurality of discrete particles containing the active ingredient. The particles so obtained were dried using conventional tray dryers/fluid bed dryers to an outlet temp. of 45° C.

### INTERMEDIATE EXAMPLE 3

A plurality of particles containing the active drug were prepared from the following materials:

| | |
|---|---|
| Non-pareil seeds | 108.00 mg |
| Active drug | 20.00 mg |
| Sucrose | 35.90 mg |
| Corn starch | 21.10 mg |
| Talcum | 2.00 mg |
| HPC-L Klucel | 1.00 mg |
| | 186.00 mg |

Water: as required.

Particles were also made of the above materials but with the addition of 3.00 mg ammonium carbonate.

Initially, the active drug, the sucrose, the corn starch, the ammonium carbonate (when present) and the talcum were blended thoroughly to yield a dusting powder. The non-pareil seeds were loaded into the centrifugal coater and then coated with the dusting powder while spraying the HPC-L Klucel (hydroxypropyl cellulose) solution. This resulted in the production of a plurality of discrete particles containing the active ingredient. The particles so obtained were dried using conventional tray dryers/fluid bed dryers to an outlet temp. of 45° C.

### INTERMEDIATE EXAMPLE 4

A plurality of tablet cores containing an active drug were prepared from the following materials:

| | |
|---|---|
| Sucrose | 80.00 mg |
| Corn Starch | 86.00 mg |
| Active drug | 20.00 mg |
| Talcum | 1.00 mg |
| Magnesium stearate | 1.00 mg |
| Gelatine | 2.00 mg |
| | 190.00 mg |

Water: as required

Particles were also made of the above materials but with the addition of 30% by weight solution of ammonia to give a pH of 8.0-9.0.

Initially the active drug was blended with the sucrose and the corn starch in a suitable mixer. The blend containing the active drug was then granulated with a solution of the gelatine binder (with the ammonia when present). The granules were dried using conventional means, then lubricated with the talcum and magnesium stearate. Finally, the granules were compressed into a suitable shape for a tablet core using conventional compression equipment.

### INTERMEDIATE EXAMPLE 5

Employing the same procedure of Intermediate Example 4, tablet cores were also made of the following composition:

| | |
|---|---|
| Sucrose | 45.00 mg |
| Dicalcium phosphate | 75.00 mg |
| Corn starch | 45.00 mg |
| Active drug | 20.00 mg |
| Talcum | 1.00 mg |
| Magnesium stearate | 2.00 mg |
| Gelatine | 2.00 mg |
| | 190.00 mg |

Water: as required

Particles were also made of the above materials but with the addition of 30% by weight solution of ammonia to a pH of 8.0-9.0.

### INTERMEDIATE EXAMPLE 6

A plurality of tablet cores containing an active drug were prepared from the following materials:

| | |
|---|---|
| Sucrose | 80.00 mg |
| Corn Starch | 86.00 mg |
| Active drug | 20.00 mg |
| Talcum | 1.00 mg |
| Magnesium stearate | 1.00 mg |
| Gelatine | 2.00 mg |
| | 190.00 mg |

Water: as required

Particles were also made of the above materials but also including 3.00 mg ammonium carbonate.

Initially the active drug was blended with the sucrose, corn starch and the ammonium carbonate (when present) in a suitable mixer. The blend containing the active drug was then granulated with a solution of the gelatine binder. The granules were dried using conventional means, then lubricated with the talcum and magnesium stearate. Finally, the granules were compressed into a suitable shape for a tablet core using conventional compression equipment.

### INTERMEDIATE EXAMPLE 7

A plurality of tablet cores containing an active drug were prepared from the following materials:

| | |
|---|---|
| Active drug | 20.00 mg |
| Mannitol | 115.50 mg |
| Polyvinylpyrrolidone K30 | 4.00 mg |
| Crospovidone | 7.00 mg |
| Magnesium stearate | 3.00 mg |
| Talcum | 1.50 mg |
| Polyethylene Glycol | 2.00 mg |
| 6000 | |
| | 153.00 mg |

Water: as required

Particles were also made of the above materials but also including 2.00 mg ammonium carbonate.

The active drug was blended with the mannitol, and then granulated with a solution of PVP-K30 containing ammonium carbonate (when present). The granules were dried using conventional means, then lubricated with the talcum, magnesium stearate, PEG 6000 and Crospovidone. Finally, the granules were compressed into a suitable shape for a tablet core using conventional compression equipment.

### INTERMEDIATE EXAMPLE 8

A plurality of tablet cores containing an active drug were prepared from the following materials:

| | |
|---|---|
| Active drug | 20.00 mg |
| Mannitol | 115.50 mg |
| Polyvinylpyrrolidone K30 | 4.00 mg |
| Crospovidone | 7.00 mg |
| Magnesium stearate | 3.00 mg |
| Talcum | 1.50 mg |
| Polyethylene Glycol | 2.00 mg |
| 6000 | |
| | 153.00 mg |

Water: as required

Particles were also made of the above materials but including 30% by weight ammonia solution to a pH of 8.0-9.0.

The active drug was blended with the mannitol. It was then granulated with the ammonia solution (when present). The granules were dried using conventional means, then lubricated with the talcum, magnesium stearate, PEG 6000 and Crospovidone. Finally, the granules were compressed into a suitable shape for a tablet core using conventional compression equipment.

### INTERMEDIATE EXAMPLE 9

190.00 mg of the particles of the composition formed in Intermediate Example 1 were treated with 3.00 mg of polydimethylsiloxane, and as much water as necessary. The coating was carried out using a conventional coating pan. Instead, it could have been carried out using a fluidised bed coater. This produced a moisture resistant coating around the particles of the composition of Intermediate Example 1.

### INTERMEDIATE EXAMPLE 10

190.00 mg of the particles of the composition formed in Intermediate Example 1 were also treated with 20.00 mg of an emulsion of 4.27 mg of polydimethylsiloxane, and 15.73 mg sucrose along with other conventional coating additives. The coating was carried out using a conventional coating pan. Instead, it could have been carried out using a fluidised bed coater. This produced a moisture resistant coating around the particles of the composition of Intermediate Example 1.

### INTERMEDIATE EXAMPLE 11

193.00 mg of the particles of the composition formed in Intermediate Example 2 were also treated with 20.00 mg of an emulsion of 4.27 mg of polydimethylsiloxane, and 15.73 mg sucrose along with other conventional coating additives. The coating was carried out using a conventional coating pan. Instead, it could have been carried out using a fluidised bed coater. This produced a moisture resistant coating around the particles of the composition of Intermediate Example 2.

### INTERMEDIATE EXAMPLE 12

189.00 mg of the particles of the composition formed in Intermediate Example 3 were treated with 3.00 mg of polydimethylsiloxane, and as much water as necessary. The coating was carried out using a conventional coating pan. Instead, it could have been carried out using a fluidised bed coater. This produced a moisture resistant coating around the particles of the composition of Intermediate Example 3.

### INTERMEDIATE EXAMPLE 13

189.00 mg of the particles of the composition formed in Intermediate Example 3 were also treated with 20.00 mg of an emulsion of 4.27 mg of polydimethylsiloxane, and 15.73 mg sucrose along with other conventional coating additives. The coating was carried out using a conventional coating pan. Instead, it could have been carried out using a fluidised bed coater. This produced a moisture resistant coating around the particles of the composition of Intermediate Example 3.

### INTERMEDIATE EXAMPLE 14

Intermediate Example 9 was repeated, using an amount of 30% by wt. ammonia solution, in addition to the polydimethylsiloxane. This produced a moisture resistant coating around the particles of the composition of Intermediate Example 1.

### INTERMEDIATE EXAMPLE 15

The 190.00 mg particles formed in Intermediate Example 4 were coated with 3.00 mg polydimethylsiloxane to produce a moisture resistant coating around each particle.

### INTERMEDIATE EXAMPLE 16

The 190.00 mg particles formed in Intermediate Example 5 were coated with 3.00 mg polydimethylsiloxane to produce a moisture resistant coating around each particle.

### INTERMEDIATE EXAMPLE 17

The 190.00 mg particles formed in Intermediate Example 4 were also treated with 20.00 mg of an emulsion of 4.27 mg of polydimethylsiloxane, and 15.73 mg sucrose along with other conventional coating additives to produce a moisture resistant coating around each particle.

### INTERMEDIATE EXAMPLE 18

The 190.00 mg particles formed in Intermediate Example 5 were also treated with 20.00 mg of an emulsion of 4.27 mg of polydimethylsiloxane, and 15.73 mg sucrose along with other conventional coating additives to produce a moisture resistant coating around each particle.

### INTERMEDIATE EXAMPLE 19

The 193.00 mg particles formed in Intermediate Example 6 were coated with 3.00 mg polydimethylsiloxane to produce a moisture resistant coating around each particle.

### INTERMEDIATE EXAMPLE 20

The 193.00 mg particles formed in Intermediate Example 6 were also treated with 20.00 mg of an emulsion of 4.27 mg of polydimethylsiloxane, and 15.73 mg sucrose along with other conventional coating additives to produce a moisture resistant coating around each particle.

### INTERMEDIATE EXAMPLE 21

The 190.00 mg particles formed in Intermediate Example 4 were coated with polydimethylsiloxane, and an amount of 30% by wt. ammonia solution, to produce a moisture resistant coating around each particle.

### INTERMEDIATE EXAMPLE 22

The 190.00 mg particles formed in Intermediate Example 5 were coated with polydimethylsiloxane, and an amount of 30% by wt. ammonia solution, to produce a moisture resistant coating around each particle.

### INTERMEDIATE EXAMPLE 23

A plurality of particles containing the active drug were prepared from the following materials:

| | |
|---|---|
| Non-pareil seeds | 108.00 mg |
| Active drug | 20.00 mg |
| HPMC | 10.00 mg |
| Polyvinylpyrrolidone | 4.00 mg |
| Talcum | 2.50 mg |
| Water | As required |
| Total | 144.50 mg |

Initially, the polyvinylpyrrolidone and HPMC were dissolved in water, to obtain a clear solution. To this solution were added the active drug and talcum, in that order, and dispersed well. This drug suspension was sprayed onto the non-pareil seeds using a fluidised bed coater to obtain drug-loaded cores. These cores were then given a moisture resistant coating in the same way as described in Intermediate Example 9 or 10. Also, an addition of 30% by weight ammonia solution can be used as in Intermediate Example 14.

### INTERMEDIATE EXAMPLE 24

A plurality of particles containing the active drug were made from the following materials:

| | |
|---|---|
| Non-pareil seeds | 95.00 mg |
| Active drug | 20.00 mg |
| Sucrose | 30.00 mg |
| Corn Starch | 30.00 mg |
| Talcum | 10.00 mg |
| Polyvinylpyrrolidone | 4.00 mg |
| HPMC | 1.00 mg |
| Water | As required |
| Total | 190.00 mg |

Particles were also made of the above materials with the addition of 30% by weight solution of ammonia to pH 8.0-9.0.

The procedure used was as in Intermediate Example 1, the PVP being included in the dusting powder.

### INTERMEDIATE EXAMPLE 25

A plurality of particles containing the active drug were prepared from the following materials:

| | |
|---|---|
| Non-pareil seeds | 108.00 mg |
| Active drug | 20.00 mg |
| Sucrose | 35.90 mg |
| Corn Starch | 21.10 mg |
| Talcum | 2.00 mg |
| Polyvinylpyrrolidone | 4.00 mg |
| HPC-L Klucel | 1.00 mg |
| Water | As required |
| Total | 192.00 mg |

Particles were also made of the above materials with the addition of 30% by weight solution of ammonia to pH 8.0-9.0.

The procedure used was as in Intermediate Example 1, the PVP being included in the dusting powder.

### INTERMEDIATE EXAMPLE 26

The compositions obtained in Intermediate Examples 1-8 and 23, 24, 25 were treated with 11.00 mg of a mixture comprising of 2.85 mg of an emulsion of polydimethylsiloxane with 9.00 mg of a binding agent as described earlier (Sucrose/Polyvinylpyrrolidone/Shellac/Xanthan Gum), along with 1 mg of talc. The coating was carried out using a fluidised bed coater. Alternately, it could have been carried out using a conventional coating pan. This produced a moisture resistant coating around each composition of the respective examples.

### EXAMPLE 1

In this Example, the particles formed in Intermediate Examples 9 to 14 were provided with an enteric coating to yield compositions according to the present invention. Some were coated with cellulose acetate phthalate, some with HPMCP and some with Eudragit L 100 55. In each case, 500.00 g of the particles were each coated with 55.00 g of the respective enteric coating polymer. The enteric coating polymer was deposited using a conventional coating process.

### EXAMPLE 2

In this Example, the particles formed in Intermediate Examples 15 to 22 were each coated with an enteric coating polymer to yield tablet compositions according to the present invention. Some were coated with cellulose acetate phthalate, some with HPMCP and some with Euragdit L 100 55. In each case, the enteric coating polymer was deposited using a conventional process for coating.

### EXAMPLE 3

500.00 g of the enteric coated particles from Example 1 were coated with 3.00 mg per unit dosage form of a moisture resistant coating of polydimethylsiloxane. The moisture resistant coating was sprayed onto the particles.

### EXAMPLE 4

500.00 g of the enteric coated particles from Example 1 were coated with 20.00 mg of an emulsion containing 4.27 mg of polydimethylsiloxane, and 15.73 mg sucrose per unit dosage form to give a moisture resistant coating. The moisture resistant coating was sprayed onto the particles.

### EXAMPLE 5

500.00 g of the enteric coated tablets from Example 2 were each coated with 3.00 mg per unit dosage form of a moisture resistant coating of polydimethylsiloxane. The moisture resistant coating was sprayed onto the tablets.

### EXAMPLE 6

500.00 g of the enteric coated tablets from Example 2 were each coated with 20.00 mg of an emulsion containing 4.27 mg of polydimethylsiloxane, and 15.73 mg sucrose per unit dosage form to give a moisture resistant coating. The moisture resistant coating was sprayed onto the tablets.

### EXAMPLE 7

The enteric coated particles of Examples 1 and 2 were respectively employed in the following formulae:

| | | |
|---|---|---|
| Particles | 193.00 | mg |
| Microcrystalline | 20.00 | mg |
| Cellulose | | |
| Starch | 50.00 | mg |
| Talcum | 1.00 | mg |
| Particles | 189.00 | mg |
| Microcrystalline | 20.00 | mg |
| Cellulose | | |
| Starch | 50.00 | mg |
| Talcum | 1.00 | mg |

The particles were intimately mixed with the other ingredients in a suitable mixer. The resultant blend was made into peltabs which were subsequently respectively provided with a moisture resistant and an enteric coating as follows.

Moisture Resistant Coatings

3.00 mg polydimethylsiloxane (optionally with 30% ammonia solution) to produce a moisture resistant coating around each particle; or

20.00 mg of an emulsion of 4.27 mg of polydimethylsiloxane, and 15.73 mg sucrose, along with other conventional coating additives to produce a moisture resistant coating around each particle.

Enteric Coatings

Enteric coatings included cellulose acetate phthalate, HPMCP and Euragdit L 100 55.

It will appreciated that modifications may be made to the invention described above.

1. A pharmaceutical composition which is a solid pellet comprising an inert core, a benzimidazole in or on the core, a moisture resistant coating around the core, the moisture resistant coating comprising at least one hydrophobic material, and an enteric coating around the moisture resistant coating.

2. A composition according to claim 1, wherein the benzimidazole is omeprazole, lansoprazole, timoprazole, pariprazole or pantoprazole.

3. A composition according to claim 2, wherein the benzimidazole is omeprazole.

4. A composition according to any of claims 1 to 3, wherein the hydrophobic material is selected from the group consisting of a polyalkylsiloxane, castor oil, mineral oil, isopropyl myristate, stearic acid and cetyl alcohol.

5. A composition according to claim 4, wherein the hydrophobic material comprises a polyalkylsiloxane.

6. A composition according to claim 5, wherein the polyalkylsiloxane is polydimethylsiloxane.

7. A pharmaceutical composition which is a solid pellet comprising an inert core, a benzimidazole in or on the core, a moisture resistant coating around the core, the moisture resistant coating comprising at least one hydrophobic material, and an enteric coating around the moisture resistant coating, wherein the benzimidazole is omeprazole, and the moisture resistant coating comprises a polyalkylsiloxane.

8. A composition according to claim 7, wherein the polyalkylsiloxane is polydimethylsiloxane.

9. A composition according to any of claims 1 to 8, wherein the moisture resistant coating further comprises at least one binding agent.

10. A composition according to claim 9, wherein the binding agent is selected from the group consisting of a sugar, polyvinyl-pyrrolidone, shellac and xanthan gum.

11. A composition according to claim 10, wherein the binding agent comprises a sugar.

12. A composition according to any of claims 1 to 11, wherein the benzimidazole in or on the core is in an alkaline environment.

13. A composition according to claim 12, wherein the benzimidazole is present as an intimate mixture with at least one alkali.

14. A composition according to claim 13, wherein the benzimidazole is present as an intimate mixture with ammonia, ammonium hydroxide or ammonium carbonate.

15. A composition according to claim 14, wherein the benzimidazole is present as an intimate mixture with ammonium carbonate.

16. A tablet which comprises a pharmaceutical composition according to any of claims 1 to 15, wherein the inert core is formed from a plurality of granules comprising the benzimidazole, which granules are compressed together to form the core.

17. A capsule which comprises a capsule shell containing a plurality of pellets as provided by a pharmaceutical composition according to any of claims 1 to 15, wherein the benzimidazole is present on the inert core.

18. A process of preparing a composition according to claim 1, which process comprises providing an inert core having a benzimidazole in or on the core, applying a moisture resistant coating around the core, the moisture resistant coating comprising at least one hydrophobic material, and applying an enteric coating around the moisture resistant coating.

19. A process according to claim 18, wherein the benzimidazole is formulated in an alkaline environment.

20. A process according to claim 19, wherein the benzimidazole is formulated in the presence of ammonia, ammonium hydroxide or ammonium carbonate.

21. A process according to claim 20, wherein the benzimidazole is formulated in the presence of ammonium carbonate.

22. A process according to any of claims 18 to 21, wherein the hydrophobic material comprises a polyalkylsiloxane.

23. A process according to claim 22, wherein the polyalkylsiloxane is polydimethylsiloxane.

24. A process according to any of claims 18 to 23, wherein the benzimidazole is omeprazole, lansoprazole, timoprazole, pariprazole or pantoprazole.

25. A process according to claim 24, wherein the benzimidazole is omeprazole.

26. A process of preparing a tablet according to claim 16, which process comprises compressing together a plurality of the granules to form the core, applying the moisture resistant coating to the core and applying the enteric coating to the moisture resistant coating.

27. A process of preparing a capsule according to claim 17, which process comprises providing the benzimidazole on the inert core, applying the moisture resistant coating thereto, applying the enteric coating to the moisture resistant coating, so as to provide a plurality of pellets as provided by a pharmaceutical composition according to claim 1, and enclosing the pellets in a capsule shell.
